(19) United States
(12) Patent Application Publication
(10) Pub. No.: US 2022/0381722 A1
(43) Pub. Date: Dec. 1, 2022

(54) SENSOR FOR DOPAMINE-SELECTIVE DETECTION AND PREPARATION METHOD THEREFOR

(71) Applicant: KOREA INSTITUTE OF SCIENCE AND TECHNOLOGY, Seoul (KR)

(72) Inventors: Yi Jae LEE, Seoul (KR); Seunghyeon KO, Seoul (KR)

(73) Assignee: KOREA INSTITUTE OF SCIENCE AND TECHNOLOGY, Seoul (KR)

(21) Appl. No.: 17/747,354

(22) Filed: May 18, 2022

(30) Foreign Application Priority Data
May 25, 2021 (KR) 10-2021-0067218

(51) Int. Cl. G01N 27/327; G01N 27/30; B82Y 30/00

(52) U.S. Cl. CPC G01N 27/3278; G01N 27/301; B82Y 30/00

(57) ABSTRACT

The present invention relates to a sensor for dopamine-selective detection, a preparation method therefor, and use thereof.

### CROSS REFERENCE TO RELATED APPLICATIONS

This application claims the benefit under 35 USC 119(a) of Korean Patent Application No. 10-2021-0067218 filed on May 25, 2021, in the Korean Intellectual Property Office, the entire disclosure of which is incorporated herein by reference for all purposes.

### TECHNICAL FIELD

The present invention relates to a sensor for dopamine-selective detection, a preparation method therefor, and use thereof.

### BACKGROUND ART

Dopamine is a neurotransmitter that has key roles in the kidneys, myocardium, and central nervous system. Dopamine is a catecholamine-based organic compound and is found in the central nervous system of various animals. Dopamine accounts for 80% of the catecholamine content in the brain and functions as a neurotransmitter, which is a compound released by neurons to send signals to other neurons. Such neurotransmitters are synthesized in specific regions of the brain, but affect many regions systemically.

The brain includes several distinct dopamine pathways, one of which has a major role in the motivational component of reward-motivated behavior. The anticipation of most reward types increases the level of dopamine in the brain, and many addictive drugs increase the dopamine release or block the dopamine reuptake into neurons after release. Other brain dopamine pathways are involved in motor control and in regulating the release of various hormones. These pathways and cell groups form a dopamine system which has neuromodulatory functions.

Outside the central nervous system, dopamine functions primarily as a local paracrine messenger. Dopamine inhibits norepinephrine release and acts as a vasodilator at normal concentrations in blood vessels. In addition, dopamine increases sodium excretion and urine output in the kidneys, reduces insulin production in the pancreas, reduces gastrointestinal motility and protects intestinal mucosa in the digestive system, and reduces the activity of lymphocytes in the immune system. Dopamine is locally synthesized in each of these peripheral systems, excluding the blood vessels, and exerts effects thereof near the cells to which dopamine is released.

Several important diseases of the nervous system are associated with dysfunctions of the dopamine system, and some of the main medications used to treat the diseases work by altering the effects of dopamine. For example, Parkinson's disease, which is a degenerative disease causing tremor and motor impairment, is caused by a loss of dopamine-secreting neurons in an area of the midbrain called the substantia nigra. As another example, schizophrenia is involved in altered levels of dopamine activity, and most antipsychotic drugs used to treat the disease are dopamine antagonists that reduce dopamine activity. Similarly, the dopamine antagonists are also some of the most effective anti-nausea agents. On the other hand, restless legs syndrome and attention deficit hyperactivity disorder (ADHD) are associated with reduced dopamine activity, and thus dopaminergic stimulants are often used to treat the diseases but may be addictive in high doses.

Although dopamine has various roles in the body as described above, an increase or decrease in the amount of dopamine release may cause important diseases, and thus the measurement of in vivo dopamine levels is an important means to prevent or treat diseases as well as monitor the progress of the diseases. However, sensors with excellent dopamine detection performance need to be developed since the levels of dopamine are very low in vivo. In particular, methods capable of selectively detecting dopamine with high sensitivity in a sample mixed with ascorbic acid and uric acid having similar oxidation potentials to dopamine are essential in the electrochemical detection.

### DISCLOSURE

### Technical Problem

The present inventors made intensive research efforts to develop a sensor for dopamine-selective detection useful for quantitative analysis due to the low detection limit and the highly linear relation of signal with concentrations, and as a result, the present inventors identified that the selective deposition of an optimized ratio of GO/PEDOT:PSS on a working electrode of a sensor, which includes the working electrode, a counter electrode, and a reference electrode, by selective electro-polymerization can attain a low detection limit of down to 0.008 μM and achieve selective qualitative and/or quantitative analysis of dopamine even in the presence of interfering substances, thereby completing the present invention.

### Technical Solution

Each description and exemplary embodiment disclosed in the present invention may also be applied to other descriptions and exemplary embodiments. That is, all combinations of various elements disclosed in the present invention fall within the scope of the present invention. Further, the scope of the present invention is not limited by the specific description below.

Furthermore, those skilled in the art will recognize or be able to ascertain, using no more than routine experimentation, many equivalents to the specific embodiments of the invention described herein. Furthermore, such equivalents are intended to be encompassed by the present invention.

In addition, throughout this specification, when a part is referred to as “comprising” an element, it will be understood that other elements may be further comprised rather than other elements being excluded unless content to the contrary is specially described.

Hereinafter, the present invention will be described in detail.

A first aspect of the present invention provides a method for preparing a dopamine-sensitive sensor, the method comprising: a first step of preparing a solution comprising graphene oxide (GO), 3,4-ethylenedioxythiophene (EDOT), and polystyrene sulfonate (PSS); and a second step of immersing, in the solution, electrodes having a reference electrode, a counter electrode, and a working electrode formed on a support, and applying a current to the working electrode to selectively deposit GO/PEDOT:PSS thereon, wherein the solution comprises 0.0003 mol to 0.0015 mol of EDOT relative to 1 g of GO, and the molar ratio of EDOT and PSS is 1:7-13.

The preparation method of the present invention can provide a dopamine-sensitive sensor with high sensitivity and/or selectivity without damage to thin-film electrodes since the GO/PEDOT:PSS layer can be selectively deposited through electro-polymerization by applying a low current of several μA to the working electrode.

For example, the second step may be performed for 200 to 500 seconds. Specifically, the second step may be performed for 250 to 400 seconds or 270 to 300 seconds, but is not limited thereto. In cases where the second step is performed for less than 200 seconds or more than 500 seconds, PEDOT:PSS nanoparticles are not uniformly distributed along the GO layer but aggregate or are non-uniformly distributed on the finally produced sensor, resulting in a remarkable reduction in charge storage capacity (CSC) and/or a remarkable increase in interfacial impedance in the sensor, causing a degradation in the performance as a sensor.

A second aspect of the present invention provides a dopamine-sensitive sensor equipped with electrodes having a reference electrode, a counter electrode, and a working electrode formed on a support, the working electrode comprising a selectively deposited GO/PEDOT:PSS layer, wherein the GO/PEDOT:PSS layer comprises 0.0003 mol to 0.0015 mol of EDOT relative to 1 g of GO, and the molar ratio of EDOT and PSS is 1:7-13.

For example, the dopamine-sensitive sensor of the present invention may be prepared by way of the method of the second aspect, but is not limited thereto. As described above, the method of the first aspect enables the introduction of the GO/PEDOT:PSS layer through a low current that causes no damage to the electrode.

A third aspect of the present invention provides a method for detecting dopamine, comprising bringing the electrodes of the sensor of the first aspect into contact with a dopamine-containing sample to perform differential pulse voltammetry (DPV).

For example, the detection method of the present invention can attain a detection limit of 0.007 μM to 0.1 μM. The detection method of the present invention can attain a detection limit of specifically 0.007 μM to 0.05 μM, and more specifically 0.075 μM to 0.01 μM, but is not limited thereto.

Furthermore, the detection method of the present invention may provide a sensitivity of 50 μA/μM·cm² to 100 μA/μM·cm². For example, the detection method of the present invention can provide a sensitivity of 60 μA/μM·cm² to 80 μA/μM·cm², and specifically 65 μA/μM·cm² to 75 μA/μM·cm², but is not limited thereto.

Also, a variation of the peak current measured by the detection method of the present invention is linearly proportional to the concentration of dopamine.

For example, compared with the above-described range, as for the ratio of GO to EDOT and PSS, a low proportion of GO significantly enhances the sensitivity of detection but increases the detection limit, and thus may enable neither quantitative analysis of low-concentration dopamine nor qualitative analysis for small amounts of samples. However, a high proportion of GO tends to gradually lower the sensitivity but decrease the detection limit, and a higher proportion of GO significantly results in a remarkable deterioration in linearity and a high detection limit. Therefore, qualitative and/or quantitative detection of dopamine can be attained by selecting the mixing ratio of GO to EDOT and PSS from a ratio range in which the sensitivity may slightly deteriorate but a comparatively low detection limit and a favorable signal linearity with concentrations are attained.

For example, the detection method of the present invention enables a selective detection of dopamine in samples mixed with ascorbic acid (AA), uric acid (UA), or both thereof. Specifically, in samples in which ascorbic acid and/or uric acid are mixed with dopamine, a change in dopamine concentration can be qualitatively and/or quantitatively detected, without interference of these interfering substances, according to the current value change at a specific potential through potential scanning.

As described above, the method for detecting dopamine using the sensor of the present invention can not only detect a trace of dopamine in a sample, but also attain qualitative as well as quantitative analysis of dopamine owing to excellent linearity in variation of the peak current according to the dopamine concentration.

A fourth aspect of the present invention provides a method of providing information for diagnosing an abnormal dopamine secretion—related disease, comprising quantitatively analyzing a sample by using the method for detecting dopamine of the third aspect, the sample being isolated from a subject suspected of abnormal dopamine secretion.

For example, the abnormal dopamine secretion—related disease that can be diagnosed by way of the information providing method of the present invention is a disease caused by reduced or increased dopamine secretion, and may be depression, schizophrenia, attention deficit/hyperactivity disorder (ADHD), psychosis, or Parkinson's disease, but is not limited thereto.

### Advantageous Effects

According to the sensor of the present invention, when GO/PEDOT:PSS is deposited on an electrode surface, a low current is applied to only the working electrode in a solution in which GO is mixed with PSS and the monomer EDOT at an optimized ratio, so that the GO/PEDOT:PSS layer can be selectively introduced on the working electrode through electro-polymerization without damage to the electrode, and the sensor thus prepared can not only detect a trace of dopamine at a low detection limit of down to 0.008 μM but can also attain quantitative analysis of dopamine at excellent selectivity and high linearity with concentrations even in the presence of interfering substances, and thus can be advantageously used to detect the dopamine concentration in a biosample, and furthermore, on the basis of these advantages, the sensor can be applied to the diagnosis of diseases caused by abnormal dopamine secretion.

### BRIEF DESCRIPTION OF DRAWINGS

FIG. 1 schematically shows a method for preparing a dopamine sensor according to an exemplary embodiment of the present invention.

FIG. 2 shows the DPV measurement results for dopamine solutions having concentrations of 0.01 μM to 0.7 μM according to the mixing ratio of graphene oxide (GO) and EDOT:PSS solutions.

FIG. 3 shows SEM images of the surfaces of the electrodes with GO/PEDOT:PSS deposition prepared for different deposition times.

FIG. 4 shows the EIS and CV measurement results of dopamine detection sensors having working electrodes with GO/PEDOT:PSS deposition prepared for different deposition times.

FIG. 5 shows an SEM image of the surface of an electrode with bare gold (Au), graphene oxide (GO), PEDOT:PSS, or GO/PEDOT:PSS deposition.

FIG. 6 shows SEM images of the surface of the electrode with GO/PEDOT:PSS deposition.

FIG. 7 shows the EIS and CV measurement results of a dopamine detection sensor having a working electrode with GO/PEDOT:PSS deposition according to an exemplary embodiment of the present invention. For comparison, a bare gold electrode or an electrode with deposition of GO or PEDOT:PSS alone was tested under the same conditions.

FIG. 8 schematically shows a driving method of a dopamine detection sensor having a working electrode with GO/PEDOT:PSS deposition according to an exemplary embodiment of the present invention.

FIG. 9 shows the DPV curves (FIG. 9A) and the peak current (FIG. 9B) for varying dopamine concentrations, and the DPV curves (FIG. 9C) and the peak current (FIG. 9D) for varying dopamine concentrations as measured in the presence of ascorbic acid and uric acid as measured by a dopamine detection sensor having a working electrode with GO/PEDOT:PSS deposition according to an exemplary embodiment of the present invention.

FIG. 10 shows the peak currents, dopamine sensitivity, linearity, and detection limit of a dopamine detection sensor having a working electrode with GO/PEDOT:PSS deposition according to an exemplary embodiment of the present invention. As comparative examples, a bare gold electrode and an electrode with GO alone deposition were used.

### DETAILED DESCRIPTION OF THE INVENTION

Hereinafter, the present invention will be described in detail with reference to exemplary embodiments. However, these exemplary embodiments are for illustrative purposes only, and the scope of the present invention is not intended to be limited by these exemplary embodiments.

### EXAMPLE 1

### Preparation of Flexible Dopamine Sensor with GO/PEDOT:PSS Composite

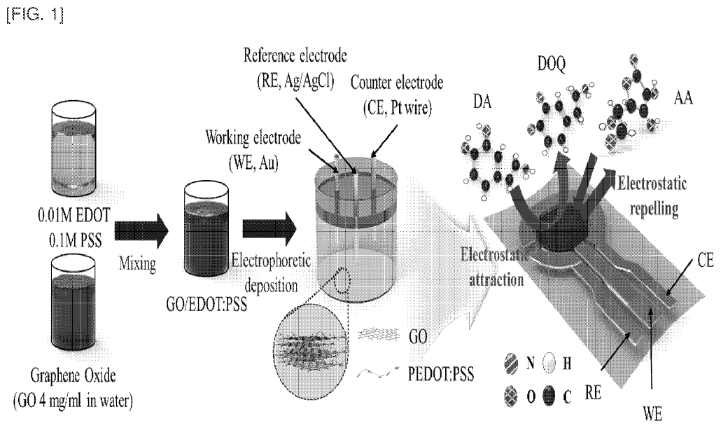

A working electrode, a counter electrode (or an auxiliary electrode), and a reference electrode were configured by patterning gold electrodes (Cr/Au=100/1000 Å thick) on a polyimide film with a thickness of about 20 μm. To prepare a GO/PEDOT:PSS electrode, a graphene oxide solution (4 mg/mL) in water and a PEDOT:PSS solution (a mixture of 0.01 M EDOT and 0.1 M PSS) were mixed at a ratio of 5:1, and uniformly mixed with vortexing. The previously prepared sensor electrodes were sufficiently immersed in the mixture solution, and then a current of 4 μA was applied to the working electrode for 300 seconds. The negatively charged GO/PEDOT:PSS was attracted to the electrode and adsorbed onto the electrode interface. It was visually confirmed that GO/PEDOT:PSS was selectively deposited in the form of a black and transparent thin film on the working electrode, and the film was dried at room temperature for 5 hours. The preparation process is schematically shown in FIG. 1.

### EXAMPLE 2

### Effects of Different Mixing Ratios in GO/PEDOT:PSS Composite

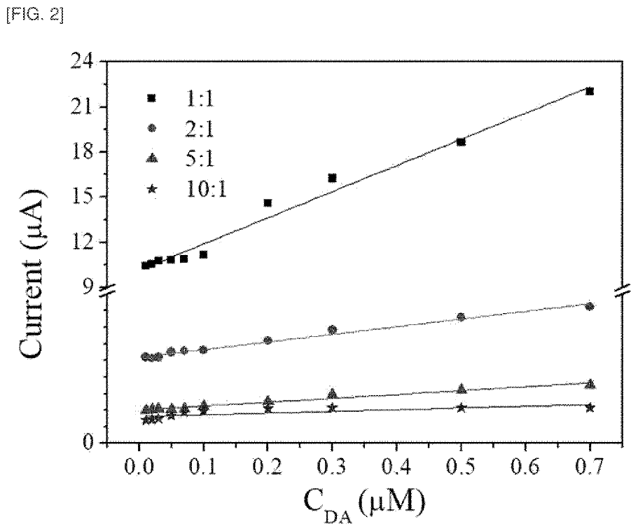

Dopamine sensors were prepared by the same method as in Example 1 except that the ratio of the graphene oxide solution (4 mg/mL) in water and the PEDOT:PSS solution (a mixture of 0.01 M EDOT and 0.1 M PSS) was changed to 1:1, 2:1, and 10:1, respectively. Then, the sensitivity, signal linearity, and detection limit of the dopamine sensors were measured and comparatively analyzed, and the results are shown in FIG. 2 and Table 1. As shown in FIG. 2 and Table 1, the sensors prepared by mixing the GO solution and the EDOT:PSS solution at ratios of 2:1 and 5:1 showed appropriate levels of sensitivity, linearity, and detection limit, and thus sensors prepared using the solutions with a ratio of 5:1 were employed in the following examples and test examples.

TABLE 1

| GO:(EDOT:PSS) | Sensitivity | Linearity | Detection limit |
|---|---|---|---|
| ratio | (μA/μM · cm²) | (R²) | (μM) |
| 1:1 | 384.82 | 0.9799 | 0.2 |
| 2:1 | 38.26 | 0.9823 | 0.1 |
| 5:1 | 17.2 | 0.9636 | 0.01 |
| 10:1 | 11.94 | 0.5397 | 0.07 |

### EXAMPLE 3

### Effects of Different Deposition Times of GO/PEDOT:PSS Composite

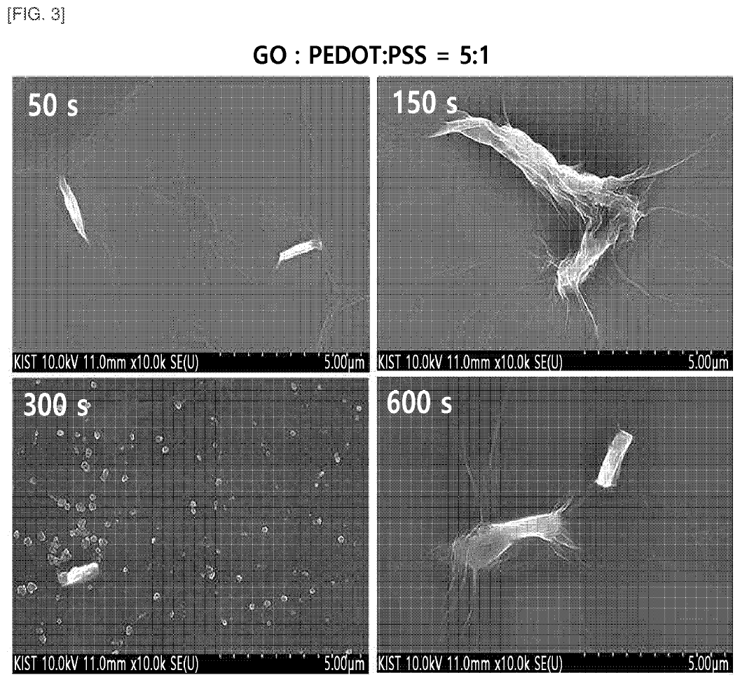

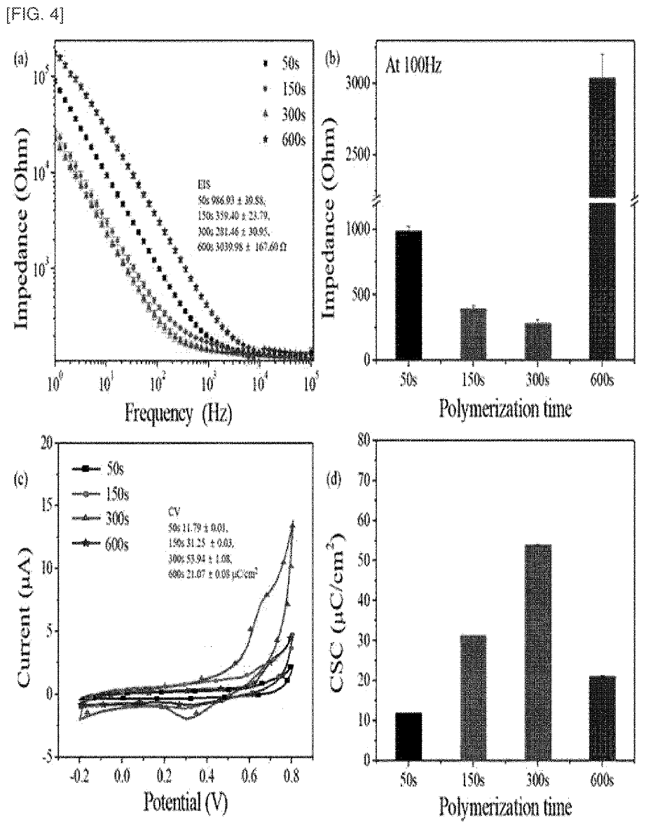

Dopamine sensors were prepared by the same method as in Example 1 except that the time of application of the current to the working electrode was changed to 50, 150, and 600 seconds, respectively. The SEM observation results of surface morphology of the working electrodes are shown in FIG. 3, and the measurement results of electrochemical impedance spectroscopy (EIS) and cyclic voltammetry (CV) characteristics are shown in FIG. 4. As shown in FIG. 3, the PEDOT:PSS nanoparticles were uniformly distributed along the GO layer on the electrode surface under a deposition time of 300 seconds. As shown in FIG. 4, the electrode under a deposition time of 300 seconds showed the lowest interfacial impedance, which was about 25% compared with that of the electrode under a deposition time of 50 seconds and less than 10% compared with that of the electrode under a deposition time of 600 seconds. On the other hand, the CSC value of the electrode under a deposition time of 300 seconds significantly increased to be at least five times that of the electrode with deposition for 50 seconds.

### Experimental Example 1

### Verification of Flexible Dopamine Sensor with GO/PEDOT:PSS Composite

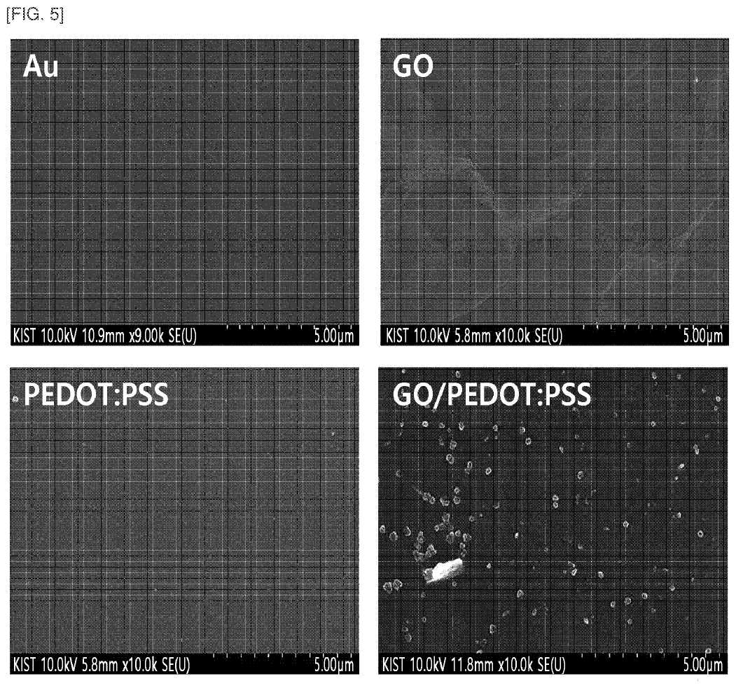

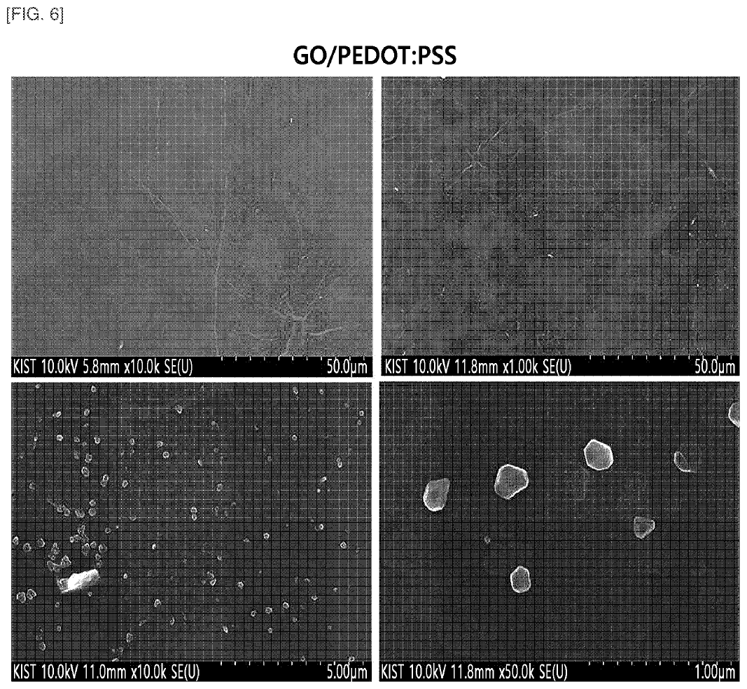

To investigate the surface morphology change by GO/PEDOT:PSS deposition on the working electrode prepared according to Example 1, SEM analysis was performed, and the results are shown in FIGS. 5 and 6. Specifically, FIG. 5 shows the observations at the same magnification of the surfaces of the electrodes with deposition of a bare gold thin film, GO, PEDOT:PSS, and GO/PEDOT:PSS composite, and FIG. 6 shows images of the electrode with GO/PEDOT:PSS deposition, measured at different magnifications. As shown in FIGS. 5 and 6, the morphology of wrinkles and/or ripples typically appearing in 2D materials was confirmed in graphene oxide (GO), and the formation of multi-layered plate-like GO and randomly distributed PEDOT:PSS particles was confirmed in GO/PEDOT:PSS.

### Experimental Example 2

### Electrical Properties of Flexible Dopamine Sensor of GO/PEDOT:PSS Composite

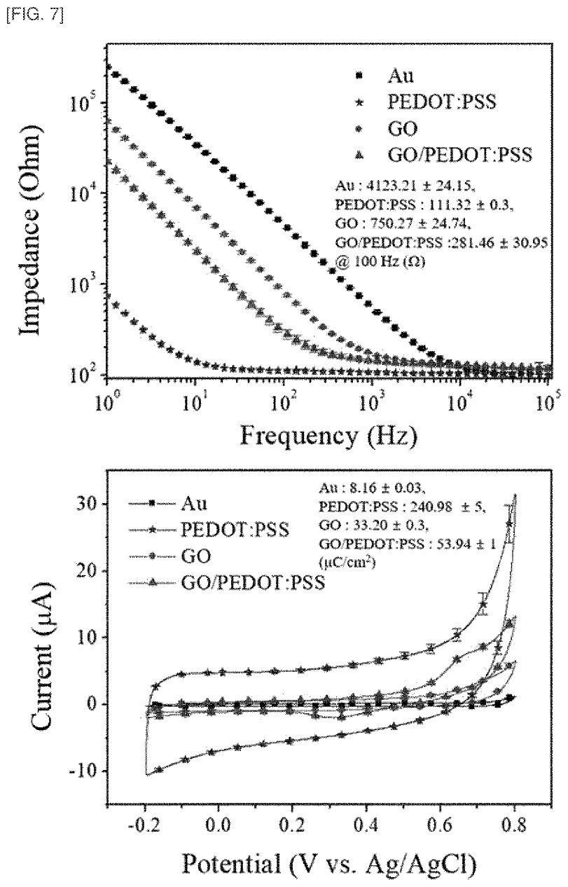

The charge storage capacity (CSC) and the impedance at the 1 kHz band of the working electrode of a GO/PEDOT:PSS composite prepared according to Example 1 were measured by cyclic voltammetry and electrochemical impedance spectroscopy, and the results are shown in FIG. 7. For comparison, the bare gold electrode and the electrodes with only GO and PEDOT:PSS depositions were also tested. As shown in FIG. 7, the working electrode of a pure GO/PEDOT:PSS composite showed low CSC and a high impedance value compared with the electrode with only PEDOT:PSS deposition, but showed a low impedance value and high CSC compared with the bare gold electrode or the electrode with GO deposition, suggesting the improvement in electrical properties through polymerization deposition with EDOT:PSS.

### Experimental Example 3

### Current Response Characteristics of Sensor Having Working Electrode of GO/PEDOT:PSS Composite to Dopamine Concentrations

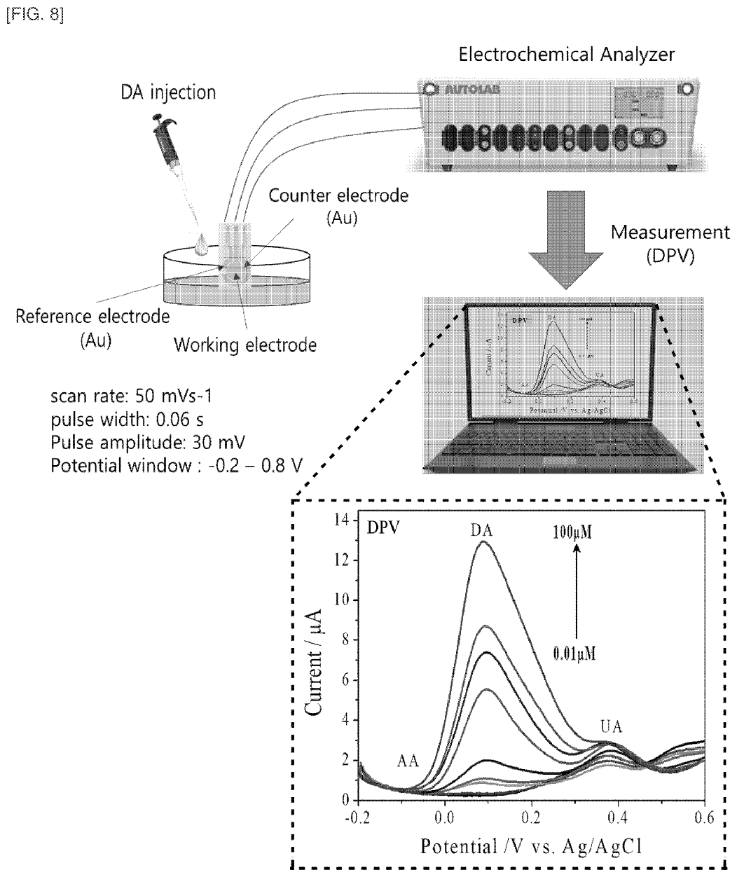

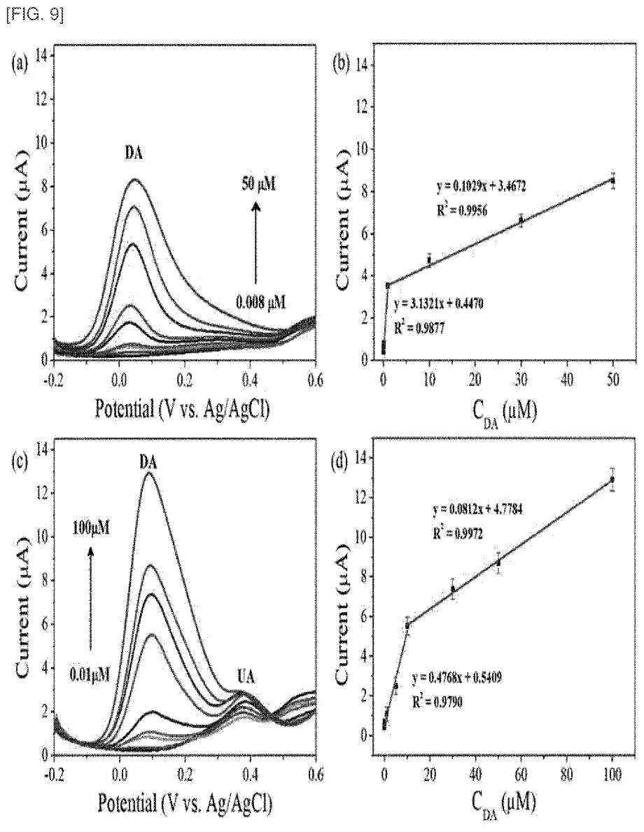

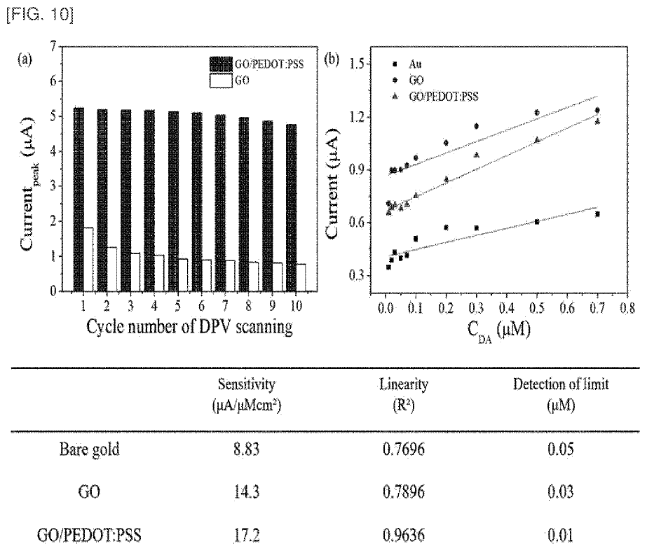

The current peak value change according to the dopamine (DA) concentration adjusted from 0.008 μM to 50 μM was measured and analyzed using a sensor having a working electrode of a GO/PEDOT:PSS composite prepared according to Example 1 by differential pulse voltammetry (DPV) with a scan rate of 50 mV/s, a pulse amplitude of 30 mV, and a pulse width of 6 ms, and the current response characteristics according to the dopamine concentration at a particular potential range were investigated. The configuration and driving conditions of the used device are shown in FIG. 8, and the measurement results are shown in FIG. 9A. In addition, the sensitivity of each electrode to dopamine detection, calculated from the measurement results, is shown in FIG. 10.

As summarized in the table at the bottom of FIG. 10, the working electrode of GO/PEDOT:PSS composite showed excellent sensitivity to dopamine (17.2 μA/μM·cm² in a range of 0.01 μM to 0.7 μM, a detection limit of 0.01 μM) compared with the bare gold electrode and the GO-adsorbed electrode, and showed a significantly improved detection limit of down to 0.008 μM and high linearity.

To investigate the selective dopamine detection performance in the co-presence of various interfering species, the dopamine detection according to the concentration was performed in an environment mixed with ascorbic acid (AA, 1 mM) and uric acid (UA, 50 μM), which are representative interfering species and of which the oxidation current peaks appear in similar bands to dopamine, and the results are shown in FIG. 9B. As shown in FIG. 9B, the sensor having a working electrode of a GO/PEDOT:PSS composite showed current responses to DA and UA at different potentials, and in particular, AA was repelled, and thus the oxidation current thereof at the electrode interface was fundamentally blocked. These results suggest that the selective detection of DA can be attained even in the presence of interfering species, such as AA or UA, leading to qualitative analysis as well as quantitative analysis without interference of interfering species.

While the present invention has been described with reference to the particular illustrative embodiments, a person skilled in the art to which the present invention pertains can understand that the present invention may be embodied in other specific forms without departing from the technical spirit or essential characteristics thereof. Therefore, the embodiments described above should be construed as exemplifying and not limiting the present disclosure. The scope of the present invention is not defined by the detailed description as set forth above but by the accompanying claims of the invention, and it should also be understood that all changes or modifications derived from the definitions and scopes of the claims and their equivalents fall within the scope of the invention.

What is claimed is:

1. A method for preparing a dopamine-sensitive sensor, the method comprising:
a first step of preparing a solution comprising graphene oxide (GO), 3,4-ethylenedioxythiophene (EDOT), and polystyrene sulfonate (PSS); and
a second step of immersing, in the solution, electrodes having a reference electrode, a counter electrode, and a working electrode formed on a support, and applying a current to the working electrode to selectively deposit GO/PEDOT:PSS thereon,
wherein the solution comprises 0.0003 mol to 0.0015 mol of EDOT relative to 1 g of GO, and the molar ratio of EDOT and PSS is 1:7-13.

2. A dopamine-sensitive sensor equipped with electrodes having a reference electrode, a counter electrode, and a working electrode formed on a support, the working electrode comprising a selectively deposited GO/PEDOT:PSS layer, wherein the GO/PEDOT:PSS layer comprises 0.0003 mol to 0.0015 mol of EDOT relative to 1 g of GO, and the molar ratio of EDOT and PSS is 1:7-13.

3. The dopamine-sensitive sensor of claim 2, being prepared by way of a first step of preparing a solution comprising graphene oxide (GO), 3,4-ethylenedioxythiophene (EDOT), and polystyrene sulfonate (PSS); and
a second step of immersing, in the solution, electrodes having a reference electrode, a counter electrode, and a working electrode formed on a support, and applying a current to the working electrode to selectively deposit GO/PEDOT:PSS thereon,
wherein the solution comprises 0.0003 mol to 0.0015 mol of EDOT relative to 1 g of GO, and the molar ratio of EDOT and PSS is 1:7-13.

4. A method for detecting dopamine, comprising bringing the electrodes of the sensor of claim 2 into contact with a dopamine-containing sample to perform differential pulse voltammetry (DPV).

5. The method for detecting dopamine of claim 4, wherein a detection limit of 0.007 μM to 0.1 μM is attained.

6. The method for detecting dopamine of claim 4, wherein a sensitivity of 50 μA/μM·cm² to 100 μA/μM·cm² is attained.

7. The method for detecting dopamine of claim 4, wherein a variation of the measured peak current is linearly proportional to the concentration of dopamine.

8. The method for detecting dopamine of claim 4, allowing of selective detection of dopamine in samples mixed with ascorbic acid (AA), uric acid (UA), or both thereof.

9. The method for detecting dopamine of claim 4, wherein qualitative or quantitative analysis of dopamine is attainable.

10. A method of providing information for diagnosing an abnormal dopamine secretion-related disease, comprising quantitatively analyzing a sample by using the method for detecting dopamine of claim 4, the sample being isolated from a subject suspected of abnormal dopamine secretion.

11. The method of providing information of claim 10, wherein the abnormal dopamine secretion-related disease is depression, schizophrenia, attention deficit/hyperactivity disorder (ADHD), psychosis, or Parkinson's disease.
